(19) United States
(12) Patent Application Publication
(10) Pub. No.: US 2025/0129000 A1
(43) Pub. Date: Apr. 24, 2025

(54) PREPARATION METHOD FOR PERFLUOROOLEFIN OLIGOMER AND APPLICATION THEREOF

(71) Applicant: Zhejiang Juhua Technology Center Co., Ltd, Zhejiang (CN)

(72) Inventors: Hongfeng LI, Zhejiang (CN); Zongling WANG, Zhejiang (CN); Liyang ZHOU, Zhejiang (CN); Shuhua WANG, Zhejiang (CN); Xiaobo XU, Zhejiang (CN)

(73) Assignee: Zhejiang Juhua Technology Center Co., Ltd, Zhejiang (CN)

(21) Appl. No.: 18/689,940

(22) PCT Filed: Aug. 16, 2022
(86) PCT No.: PCT/CN2022/112748

§ 371 Date: Mar. 7, 2024

(30) Foreign Application Priority Data
Sep. 7, 2021 (CN) 202111045203.9

(51) Int. Cl. C07C 17/278

(52) U.S. Cl. CPC C07C 17/278

(57) ABSTRACT

The present invention relates to the technical field of the fluorine chemical industry, and disclosed are a preparation method for a perfluoroolefin oligomer and an application thereof. The preparation method comprises: selecting a combination of two or more raw material monomers selected from among the following (i) and (ii) undergoing an oligomerization reaction under the catalysis of a metal fluoride salt to obtain a perfluoroolefin oligomer product the total carbon number of which is not less than 7, wherein (i) is tetrafluoroethylene, a tetrafluoroethylene oligomer or a mixture thereof, and (ii) is hexafluoropropylene, a hexafluoropropylene oligomer or a mixture thereof. The present application solves the difficult problem of high-selectivity preparation of perfluoroolefin oligomers having high carbon numbers.

### CROSS-REFERENCE TO RELATED APPLICATIONS

The application claims the benefit of the Chinese patent application No. “202111045203.9”, filed on Sep. 7, 2021, the content of which is specifically and entirely incorporated herein by reference.

### FIELD

The application relates to the technical field of the fluorine chemical industry, in particular to a preparation method for a perfluoroolefin oligomer and a use thereof.

### BACKGROUND

The perfluoroolefin is a fluorine-containing organic intermediate with excellent properties due to its carbon-carbon double bond structure, it has widespread applications, and a low global warming potential value owing to its short environmental lifetime, thus it is a novel and ideal substitute for chlorofluorocarbons (CFCs). The perfluoroolefin with carbon-carbon double bonds at the terminal groups is generally used for copolymerization of polymers, while the perfluoroolefin with carbon-carbon double bonds at the non-terminal groups can be used for preparing various derivatives, such as fluorocarbon surfactants, fluorine-containing ketones, fluorine-containing ethers, and other fluorocarbon compounds.

The commonly used perfluoroolefin at present is a perfluoroolefin oligomer prepared by using tetrafluoroethylene or hexafluoropropylene as the monomer, the perfluoroolefin oligomer mainly comprises a tetrafluoroethylene oligomer with a polymerization degree of 3-6, a hexafluoropropylene dimer, and a hexafluoropropylene trimer. The perfluoroolefin oligomer with a longer carbon chain can hardly be synthesized, the conversion rate is low, and the preparation process is accompanied by many side reactions, thus it is difficult to prepare the long-chain perfluoroolefin oligomer with a high selectivity.

### SUMMARY

Because of the above conditions, the present application aims to provide a preparation method for a perfluoroolefin oligomer and a use thereof, the present application solves the difficult problem of high-selectivity preparation of perfluoroolefin oligomers having high carbon numbers.

In order to solve the above problems, the inventors have discovered that the perfluoroolefin oligomer having an appropriate carbon chain length can be prepared with high selectivity by using a combination of tetrafluoroethylene or its oligomers and hexafluoropropylene or its oligomers as raw material monomers, and adjusting the reaction conditions of an oligomerization reaction to control the polymerization degree of the perfluoroolefin raw material monomers.

The preparation method provided by the present application can be used for preparing a perfluoroolefin oligomer containing two or more olefin structures, the perfluoroolefin oligomer has multiple branched chain structures and can be applied in multipurpose use.

To fulfill the purpose, the present disclosure mainly provides the following technical schemes:

The embodiments of the present application provide a method of preparing a perfluoroolefin oligomer, comprising: subjecting a combination of two or more raw material monomers selected from the following ingredients (i) and (ii) to an oligomerization reaction under the catalysis of a metal fluoride salt to obtain a perfluoroolefin oligomer product with the total carbon number not less than 7, wherein the ingredient (i) is tetrafluoroethylene, a tetrafluoroethylene oligomer or a mixture thereof, and the ingredient (ii) is hexafluoropropylene, a hexafluoropropylene oligomer or a mixture thereof.

Preferably, the tetrafluoroethylene oligomer is at least one selected from the group consisting of a tetrafluoroethylene dimer, a tetrafluoroethylene trimer, a tetrafluoroethylene tetramer, a tetrafluoroethylene pentamer, a tetrafluoroethylene hexamer, and a tetrafluoroethylene heptamer; the hexafluoropropylene oligomer is at least one selected from the group consisting of a hexafluoropropylene dimer and a hexafluoropropylene trimer.

Preferably, the perfluoroolefin oligomer is an olefin compound represented by the following general structural formula:

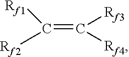

wherein R_{f1}, R_{f2}, R_{f3} and R_{f4} are independently selected from F, or perfluoroalkyl groups with 1-8 carbon atoms.

Preferably, the perfluoroolefin oligomer is an olefin compound represented by the following general structural formula:

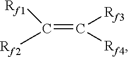

wherein R_{f1}, R_{f2}, R_{f3}, R_{f4} are independently selected from F, —CF₃, —CF₂CF₃, —CF(CF₃)₂, —C(CF₃)₃, —CF₂CF₂CF₃, —CF₂CF₂CF₂CF₃, —CF₂CF(CF₃)₂, —C(CF₂CF₃)₂CF₃, —C(CF₂CF₃)₃ or —C(CF₃)₂CF(CF₃)₂ groups.

Preferably, the oligomerization reaction is carried out in an aprotic polar solvent under the reaction temperature condition of 60-200° C.

Preferably, the aprotic polar solvent is selected from ethylene glycol dimethyl ether, diethylene glycol dimethyl ether, triethylene glycol dimethyl ether, tetraethylene glycol dimethyl ether, dimethylformamide, dimethylacetamide, acetonitrile, or a mixture thereof.

Preferably, the ratio of the total mass of raw material monomers to the mass of metal fluoride salt is 1:(0.01-0.5).

Preferably, the metal fluoride salt is one or more selected from the group consisting of cesium fluoride, potassium fluoride, and sodium fluoride.

Preferably, the reaction system of oligomerization reaction further comprises a phase transfer catalyst selected from quaternary ammonium salts, crown ether catalysts, or a mixture thereof.

Preferably, the mass fraction of said phase transfer catalyst in the reaction feedstock is not higher than 30%.

Preferably, the preparation method uses tetrafluoroethylene and/or tetrafluoroethylene dimer and hexafluoropropylene and/or hexafluoropropylene dimer as a combination of raw material monomers for carrying out an oligomerization reaction to prepare a perfluoroolein oligomer with 7-16 carbon atoms, the reaction temperature is within the range of 60-100° C., the reaction time is within the range of 2-12 h, the ratio of the total mass of raw material monomers to the mass of metal fluoride salt is 1:(0.02-0.1), the phase transfer catalyst is benzo 18-crown-6, 4-aminobenzyl-15-crown-5, 1-aza-18-crown-5, bicyclohexane-18-crown-6, or 1-aza-15-crown-5, and the mass fraction of said phase transfer catalyst in the reaction raw materials is not more than 10%.

The embodiments of the application also provide use of the perfluoroolefin oligomer prepared with the aforementioned preparation method in fluorocarbon surfactants, electronic product cleaning agents, leakage detection liquids, inert media for the polymerization reaction or diluents.

One or more technical schemes provided in the embodiments of the present application at least produce the following technical effects or advantages:

The present application uses a combination of tetrafluoroethylene or its oligomers and hexafluoropropylene or its oligomers as raw material monomers, and can prepare a perfluoroolefin oligomer with carbon number not less than 7 with high selectivity by adjusting the reaction conditions, so as to prepare a perfluoroolefin having a certain high carbon number with high selectivity, or prepare a perfluoroolefin mixture with a certain boiling range at high selectivity for direct application, or prepare a perfluoroolefin mixture with a wider boiling range, then put into use after separation, thereby solving the difficult problem of high-selectivity preparation of perfluoroolefin oligomers having high carbon numbers; the preparation method has the advantages that the process is simple, economical and environmentally friendly, the perfluoroolefin oligomer having high carbon numbers can be used as fluorocarbon surfactants, electronic product cleaning agents, leakage detection liquids, inert media for polymerization reaction or diluents, it has a significant prospect of industrial application.

### DESCRIPTION OF THE PREFERRED EMBODIMENT

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point values of the various ranges, and the individual point values may be combined with one another to produce one or more new numerical ranges, which should be deemed have been specifically disclosed herein.

In the field of fluorocarbon application, the commonly used perfluoroolefin is a tetrafluoroethylene oligomer with a polymerization degree of 3-6, a hexafluoromethylene dimer, and a hexafluoromethylene trimer, but the preparation of a tetrafluoroethylene oligomer or a hexafluoromethylene oligomer with longer carbon chain is relatively difficult, the conversion rate is low, and the preparation process is accompanied with many side reactions, thus it is difficult to prepare the long-chain perfluoroolefin oligomer with high selectivity.

To solve the above problems, the present application can be used for preparing a perfluoroolefin oligomer having a high carbon chain length with high selectivity by using a combination of tetrafluoroethylene or its oligomers and hexafluoropropylene or its oligomers as raw material monomers, and adjusting the reaction conditions of an oligomerization reaction to control the polymerization degree of the perfluoroolefin raw material monomers.

Specifically, the present application provides a method of preparing a perfluoroolefin oligomer comprising: subjecting a combination of two or more raw material monomers selected from the following ingredients (i) and (ii) to an oligomerization reaction under the catalysis of an metal fluoride salt to obtain a perfluoroolefin oligomer product with the total carbon number not less than 7, wherein the ingredient (i) is tetrafluoroethylene, a tetrafluoroethylene oligomer or a mixture thereof, and the ingredient (ii) is hexafluoropropylene, a hexafluoropropylene oligomer or a mixture thereof.

In some preferred embodiments of the present application, the tetrafluoroethylene oligomer is at least one selected from the group consisting of a tetrafluoroethylene dimer, a tetrafluoroethylene trimer, a tetrafluoroethylene tetramer, a tetrafluoroethylene pentamer, a tetrafluoroethylene hexamer, and a tetrafluoroethylene heptamer; the hexafluoropropylene oligomer is at least one selected from the group consisting of a hexafluoropropylene dimer and a hexafluoropropylene trimer.

In order to obtain perfluoroolein oligomers with different olefin structures and various carbon numbers, the types of raw material monomers can be combined voluntarily, and different monomers can be mixed according to the random fraction of dosage. For example, the combination of raw material monomers may be: a combination of a tetrafluoethylene and a hexafluopropylene, a combination of a tetrafluoethylene and a hexafluopropylene dimer, a combination of a tetrafluoethylene and a hexafluopropylene trimer, a combination of a tetrafluoethylene dimer and a hexafluopropylene, a combination of a tetrafluoethylene dimer and a hexafluopropylene dimer, a combination of a tetrafluoethylene dimer and a hexafluopropylene trimer, a combination of a tetrafluoethylene trimer and a hexafluopropylene, a combination of a tetrafluoethylene trimer and a hexafluopropylene dimer, a combination of a tetrafluoethylene trimer and a hexafluopropylene trimer; a combination of a pentafluoethylene pentamer and a hexafluopropylene, a combination of a pentafluoethylene pentamer and a hexafluopropylene dimer, a combination of a pentafluoethylene pentamer and a hexafluopropylene trimer; a combination of a pentafluoethylene heptamer and a hexafluopropylene, a combination of a pentafluoethylene heptamer and a hexafluopropylene dimer, a combination of a pentafluoethylene heptamer and a hexafluopropylene trimer; a combination of a tetrafluoroethylene, a tetrafluoroethylene dimer and a hexafluoropropylene; a combination of a tetrafluoroethylene, a hexafluoropropylene and a hexafluoropropylene dimer; a combination of a tetrafluoroethylene, a hexafluoropropylene and a hexafluoropropylene trimer; a combination of a tetrafluoroethylene, a tetrafluoroethylene trimer and a hexafluoropropylene, etc. More preferably, the tetrafluoroethylene or/and tetrafluoroethylene dimer and the hexafluoropropylene or/and the hexafluoropropylene dimer are used as a combination of raw material monomers, including a combination of a tetrafluoroethylene and a hexafluoropropylene, a combination of a tetrafluoroethylene and a hexafluoropropylene dimer, a combination of a tetrafluoroethylene dimer and a hexafluoropropylene, a combination of a tetrafluoroethylene dimer and a hexafluoropropylene dimer; a combination of a tetrafluoroethylene, a hexafluoropropylene, and a hexafluoropropylene dimer; a combination of a tetrafluoroethylene, a hexafluoropropylene, and a tetrafluoroethylene dimer; a combination of a tetrafluoroethylene, a tetrafluoroethylene dimer, and a hexafluoropropylene dimer. In the telomerization reaction process of using a tetrafluoroethylene or a tetrafluoroethylene dimer as raw material monomers and then reacting with a hexafluoropropylene, a hexafluoropropylene dimer or a hexafluoropropylene trimer, the tetrafluoroethylene will initially carry out an auto-polymerization or be polymerized with the tetrafluoroethylene dimer to form a tetrafluoroethylene oligomer with a polymerization degree of 2-7, and then perform the reaction with a hexafluoropropylene dimer or a hexafluoropropylene trimer. In the reaction process, the temperature has a large influence on different carbon chain lengths, products of five carbons and below are fundamentally obtained at low temperatures, the long-chain products s cannot be obtained; a high polymer of tetrafluoroethylene is fundamentally obtained at an excessively high temperature, thus the chain length of the tetrafluoroethylene oligomer is controlled by adjusting the reaction temperature, such that the selectivity of perfluoroolefin products with carbon number within a certain range is improved.

The preparation method provided by the application is suitable for preparing the olefin compound represented by the following general structural formula:

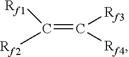

wherein R_{f1}, R_{f2}, R_{f3} and R_{f4} are independently selected from F, or perfluoroalkyl groups with 1-8 carbon atoms.

The preparation method provided by the present application is more suitable for preparing the olefin compound represented by the following general structural formula:

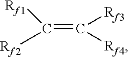

wherein R_{f1}, R_{f2}, R_{f3}, R_{f4} are independently selected from F, —CF₃, —CF₂CF₃, —CF(CF₃)₂, —C(CF₃)₃, —CF₂CF₂CF₃, —CF₂CF₂CF₂CF₃, —CF₂CF(CF₃)₂, —C(CF₂CF₃)₂CF₃, —C(CF₂CF₃)₃ or —C(CF₃)₂CF(CF₃)₂ groups.

The preparation method provided by the application is more suitable for preparing perfluoroolefin oligomer with 7-16 carbon atoms.

In some preferred embodiments of the present application, the oligomerization reaction in the preparation method is carried out in an aprotic polar solvent under the reaction temperature condition of 60-200° C. The aprotic polar solvent is preferably ethylene glycol dimethyl ether, diethylene glycol dimethyl ether, triethylene glycol dimethyl ether, tetraethylene glycol dimethyl ether, dimethylformamide, dimethylacetamide, acetonitrile, or a mixture thereof. The reaction temperature has a significant influence on the structure and distribution of the products of the perfluoroolefin oligomerization reaction, when the reaction temperature is too high, more byproducts are generated, so that the selectivity of the target product is reduced; if the reaction temperature is too low, the reaction rate is low, the raw material conversion is incomplete, and the yield of the target product is reduced. The various reaction temperatures may be adopted for the different combinations of raw material monomers from the perspective of increasing the conversion rate of raw material monomers and selectivity of perfluoroolefin oligomer product. The reaction temperature is preferably within the range of 60-100° C. The reaction time is preferably within the range of 2-12 hours.

In some preferred embodiments of the present application, the ratio of the total mass of raw material monomers to the mass of metal fluoride salt in the preparation method is 1:(0.01-0.5). More preferably, the ratio of the total mass of raw material monomers to the mass of metal fluoride salt is 1:(0.02-0.1).

In some preferred embodiments of the present application, the metal fluoride salt is one or more selected from the group consisting of cesium fluoride, potassium fluoride, and sodium fluoride.

In some preferred embodiments of the present application, the reaction system of oligomerization reaction further comprises a phase transfer catalyst; the phase transfer catalyst is preferably quaternary ammonium salts, crown ether catalysts, or a mixture thereof. The quaternary ammonium salt may be selected from methyl trialkyl (C₈-C₁₀) ammonium chloride, methyl trialkyl (C₈-C₁₀) ammonium fluoride, or (C₈H₁₇)₃N+CH₃—OSO₃CH₃; the crown ether catalyst may be selected from 4-aminobenzyl-15-crown-5, 1-aza-12-crown-5, 1-aza-15-crown-5, 1-aza-18-crown-5, bis [(benzo-15-crown-5)-15-ylmethyl]pimelate, bicyclohexane-18-crown-6, 4-formylbenzo-15-crown-5, 2-(hydroxymethyl)-15-crown-5, 4-nitrobenz-15-crown-5, poly [(dibenzo-18-crown-6)-co-formaldehyde], benzo 18-crown-6, dibenzo 18-crown-6, 18-crown-6, or a mixture thereof. More preferably, the transfer is benzo phase catalyst 18-crown-6, 4-aminobenzyl-15-crown-5, 1-aza-18-crown-5, bicyclohexane-18-crown-6, or 1-aza-15-crown-5.

In some preferred embodiments of the present application, the mass fraction of said phase transfer catalyst in the reaction feedstock is not higher than 30%. More preferably, the mass fraction of said phase transfer catalyst in the reaction feedstock is not higher than 10%.

Specifically, the preparation steps of the perfluoroolefin oligomer are as follows:
when the raw material monomers contain gaseous monomers, firstly adding non-gaseous monomers in raw materials, a metal fluoride salt, a phase transfer catalyst, and an auxiliary agent (an auxiliary agent may be not added according to actual conditions) into a reaction kettle (if all the raw materials are gaseous monomers, only adding a metal fluoride salt, a phase transfer catalyst and an auxiliary agent into the reaction kettle), removing oxygen gas in the reaction kettle, stirring the ingredients and heating to a reaction temperature, and then introducing the gaseous monomers for performing a reaction; after the reaction is finished, carrying out a liquid separation treatment to obtain a fluorine phase layer, and implementing a fractional distillation and purification to obtain a perfluoroolefin oligomer product with high purity;
when raw material monomers do not contain gaseous monomers, adding monomers in raw materials, a metal fluoride salt, a phase transfer catalyst, and an auxiliary agent (an auxiliary agent may be not added according to actual conditions) into a reaction kettle, removing oxygen gas in the reaction kettle, stirring the ingredients and heating to a reaction temperature for performing a reaction; after the reaction is finished, carrying out a liquid separation treatment to obtain a fluorine phase layer, and implementing a fractional distillation and purification to obtain a perfluoroolefin oligomer product with high purity.

Because the reaction product is an organic fluorine phase, which is generally immiscible with a solvent, a catalyst, and a phase transfer catalytic promoter in the reaction system, the reaction product can be separated through the conventional process of standing still and layering, if the reaction product is subjected to conventional operation of washing or rectification, a target product with a higher purity can be obtained.

The preparation method provided by the application can be used for preparing perfluoroolefin with a carbon number not less than 7 with high selectivity by adjusting the reaction conditions, which can not only prepare a perfluoroolefin with a certain high carbon number with high selectivity, but also prepare a perfluoroolefin mixture with a certain boiling range with high selectivity for direct application, or prepare a perfluoroolefin mixture with wider boiling range, which can be separated and then applied.

The perfluoroolefin oligomer having high carbon numbers, prepared through the preparation method, has hydrophobicity and lipophobicity, as well as chemical inertness, hydrolysis stability and thermal stability, and can be applied in many fields, such as fluorocarbon surfactants, solvents for precision or metal cleaning of electronic products (e.g. discs or circuit boards), leakage detection liquids, or the foam porosity conditioning agent in the manufacture of foam insulation (e.g., thermoplastic foams of polyurethane, phenolic resins, etc.), carrier fluids or solvents for literature or specimen preservation materials and lubricants, inert media for the polymerization reaction or diluents, a polishing abrasive for removing polishing abrasive compounds from a polished surface (e.g., metal), the perfluoroolefin oligomer prepared through the preparation method has an azeotropy with water, and can be used as a displacement drying agent for water removal, for example, removing water from jewelry or metal parts; or used as an anti-color developing agent in the traditional circuit manufacturing technology, and the like.

In order to facilitate comprehension of the scheme in the present application by those skilled in the art, the scheme of the present application is further described with reference to specific examples, and it should be understood that the examples of the present application merely serve to illustrate the scheme of the present application, instead of imposing a limitation on the protection scope of the present application.

Unless otherwise indicating the specific conditions, the following examples and comparative examples were implemented under conventional conditions. Each of the reagents or instruments used herein pertains to the conventional and commercially available products, unless specifying the manufacturers.

### Example 1

50 g of diethylene glycol dimethyl ether, 1.5 g (0.035 mol) of sodium fluoride, and 0.15 g of a phase transfer catalyst benzo 18-crown-6 were mixed and added into a reaction kettle, oxygen gas in the system was discharged, the ingredients were stirred and the temperature was raised to 60° C., a mixed gas consisting of 20 g (0.2 mol) of tetrafluoroethylene and 30 g (0.2 mol) of hexafluoropropylene was introduced in the total amount to carry out a reaction for 3 hours. After the reaction was completed, a liquid separation treatment was performed to obtain a fluorine phase layer at the lower layer, which was subjected to a gas chromatography (GC) test, the dissolved tetrafluoroethylene and hexafluoropropylene were completely converted, wherein the selectivity of C₇-C₁₆ perfluoroolefin oligomer was 94%.

### Example 2

30 g (0.1 mol) of hexafluoropropylene dimer, 1.5 g (0.026 mol) of potassium fluoride, 50 g of triethylene glycol dimethyl ether, and 5 g of a phase transfer catalyst 4-aminobenzyl-15-crown-5 were added into a reaction kettle, oxygen gas in the system was discharged, the temperature of the reaction kettle was then controlled at 70° C., a total amount of 20 g (0.2 mol) of tetrafluoroethylene was introduced into the reaction kettle, and the reaction kettle was continuously stirred for 10 hours after the introduction was finished. After the reaction was completed, a liquid separation treatment was performed to obtain a fluorine phase layer, which was subjected to a gas chromatography (GC) test, the conversion rate of the hexafluoropropylene dimer was 82%, and the selectivity of the C₈-C₁₂ perfluoroolefin oligomer was 89%.

### Example 3

7.5 g (0.0375 mol) of tetrafluoroethylene dimer, 15 g (0.05 mol) of hexafluoroethylene dimer, 3 g (0.05 mol) of potassium fluoride, 50 g of acetonitrile and 7 g of a phase transfer catalyst 1-aza-18-crown-5 were added into a reaction kettle, oxygen gas in the system was discharged, the temperature of the reaction kettle was then controlled at 100° C., a total amount of 90 g (0.6 mol) of hexafluoropropylene was introduced into the reaction kettle, and the reaction kettle was continuously stirred for 2 hours after the introduction was finished. After the reaction was completed, a liquid separation treatment was performed to obtain a fluorine phase layer, which was subjected to a gas chromatography (GC) test, the total conversion rate of the tetrafluoroethylene dimer and hexafluoropropylene dimer was 77%, and the selectivity of the C₇-C₁₄ perfluoroolefin oligomer was 95%.

### Example 4

15 g (0.075 mol) of tetrafluoroethylene dimer, 5 g (0.033 mol) of cesium fluoride, 50 g of acetonitrile and 1 g of a phase transfer catalyst bicyclohexane-18-crown-6 were added into a reaction kettle, oxygen gas in the system was discharged, the temperature of the reaction kettle was then controlled at 80° C., a total amount of 90 g (0.6 mol) of hexafluoropropylene was introduced into the reaction kettle, and the reaction kettle was continuously stirred for 4 hours after the introduction was finished. After the reaction was completed, a liquid separation treatment was performed to obtain a fluorine phase layer, which was subjected to a GC test, the conversion rate of the tetrafluoroethylene dimer was 95%, and the selectivity of the C₇-C₁₃ perfluoroolefin oligomer was 96%.

### Example 5

30 g (0.1 mol) of hexafluoropropylene dimer, 3 g (0.052 mol) of potassium fluoride, 50 g of dimethylformamide, and 5 g of a phase transfer catalyst 1-aza-15-crown-5 were added into a reaction kettle, oxygen gas in the system was discharged, the temperature of the reaction kettle was then controlled at 90° C., a total amount of 20 g (0.2 mol) of tetrafluoroethylene and 30 g (0.2 mol) of hexafluoropropylene was introduced into the reaction kettle, and the reaction kettle was continuously stirred for 5 hours after the introduction was finished. After the reaction was completed, a liquid separation treatment was performed to obtain a fluorine phase layer, which was subjected to a GC test, the conversion rate of the hexafluoropropylene dimer was 95%, and the selectivity of the C₈-C₁₆ perfluoroolefin oligomer was 92%.

### Example 6

15 g (0.075 mol) of tetrafluoroethylene dimer, 5 g (0.033 mol) of cesium fluoride, 50 g of acetonitrile, 2 g of a phase transfer catalyst 1-aza-15-crown-5 were added into a reaction kettle, oxygen gas in the system was discharged, the temperature of the reaction kettle was then controlled at 80° C., a total amount of 20 g (0.2 mol) of tetrafluoroethylene and 30 g (0.2 mol) of hexafluoropropylene was introduced into the reaction kettle, and the reaction kettle was continuously stirred for 3 hours after the introduction was finished. After the reaction was completed, a liquid separation treatment was performed to obtain a fluorine phase layer, which was subjected to a GC test, the conversion rate of the tetrafluoroethylene dimer was 85%, and the selectivity of the C₇-C₁₆ perfluoroolefin oligomer was 94%.

Finally, it should be noted that the examples merely serve to illustrate the technical solution of the present disclosure, instead of imposing limitations thereon; although the present disclosure has been described in detail with reference to the preferred embodiments, it should be understood by those skilled in the art that various modifications or equivalent substitution can be performed on the technical solution of the present disclosure, each of said modifications or equivalent substitution without departing from the spirit and scope of the technical solution of the present disclosure shall be deemed to have been contained in the protection scopes of claims of the present disclosure.

1-12. (canceled)

13. A method of preparing a perfluoroolefin oligomer comprising: subjecting a combination of two or more raw material monomers selected from the following ingredients (i) and (ii) to an oligomerization reaction under the catalysis of a metal fluoride salt to obtain a perfluoroolefin oligomer product with the total carbon number not less than 7, wherein the ingredient (i) is tetrafluoroethylene, a tetrafluoroethylene oligomer or a mixture thereof, and the ingredient (ii) is hexafluoropropylene, a hexafluoropropylene oligomer or a mixture thereof.

14. The method according to claim 13, wherein the combination of two or more raw material monomers includes at least one (i) and at least one (ii).

15. The method according to claim 13, wherein the tetrafluoroethylene oligomer is at least one selected from the group consisting of a tetrafluoroethylene dimer, a tetrafluoroethylene trimer, a tetrafluoroethylene tetramer, a tetrafluoroethylene pentamer, a tetrafluoroethylene hexamer, and a tetrafluoroethylene heptamer; the hexafluoropropylene oligomer is at least one selected from the group consisting of a hexafluoropropylene dimer and a hexafluoropropylene trimer.

16. The method according to claim 13, wherein the perfluoroolefin oligomer is an olefin compound represented by the following general structural formula:
Error! Objects cannot be created from editing field codes.,
wherein R_{f1}, R_{f2}, R_{f3} and R_{f4} are independently selected from F, or perfluoroalkyl groups with 1-8 carbon atoms.

17. The method according to claim 13, wherein the perfluoroolefin oligomer is an olefin compound represented by the following general structural formula:
Error! Objects cannot be created from editing field codes.,
wherein R_{f1}, R_{f2}, R_{f3}, R_{f4} are independently selected from F, —CF₃, —CF₂CF₃, —CF(CF₃)₂, —C(CF₃)₃, —CF₂CF₂CF₃, —CF₂CF₂CF₂CF₃, —CF₂CF(CF₃)₂, —C(CF₂CF₃)₂CF₃, —C(CF₂CF₃)₃ or —C(CF₃)₂CF(CF₃)₂ groups.

18. The method according to claim 13, wherein the reaction temperature is within the range of 60-200° C.

19. The method according to claim 13, the method comprises: the oligomerization reaction is carried out in an aprotic polar solvent under the reaction temperature condition of 60-200° C.

20. The method according to claim 19, wherein the aprotic polar solvent is selected from ethylene glycol dimethyl ether, diethylene glycol dimethyl ether, triethylene glycol dimethyl ether, tetraethylene glycol dimethyl ether, dimethylformamide, dimethylacetamide, acetonitrile, or a mixture thereof.

21. The method according to claim 13, wherein the ratio of the total mass of raw material monomers to the mass of metal fluoride salt is 1:(0.01-0.5).

22. The method according to claim 13, wherein the metal fluoride salt is one or more selected from the group consisting of cesium fluoride, potassium fluoride, and sodium fluoride.

23. The method according to claim 13, wherein the preparation method uses tetrafluoroethylene and/or tetrafluoroethylene dimer and hexafluoropropylene and/or hexafluoropropylene dimer as a combination of raw material monomers for carrying out an oligomerization reaction to prepare a perfluoroolein oligomer with 7-16 carbon atoms.

24. The method according to claim 13, wherein the reaction temperature is within the range of 60-100° C., the reaction time is within the range of 2-12 h.
